(19) United States
(12) Patent Application Publication
(10) Pub. No.: US 2022/0171879 A1
(43) Pub. Date: Jun. 2, 2022

(54) PRIVACY CONTROLS FOR MANAGING GROUP TELEHEALTH SESSIONS

(71) Applicant: Therapia Software LLC, Peachtree City, GA (US)

(72) Inventor: Shailesh Manubhai PATEL, Peachtree City, GA (US)

(73) Assignee: Therapia Software LLC, Peachtree City, GA (US)

(21) Appl. No.: 17/538,704

(22) Filed: Nov. 30, 2021

Related U.S. Application Data
(60) Provisional application No. 63/119,373, filed on Nov. 30, 2020.

(51) Int. Cl. G06F 21/62; G16H 40/67; G16H 10/60

(52) U.S. Cl. CPC G06F 21/6254; G16H 10/60; G16H 40/67

(57) ABSTRACT

Disclosed herein are system, method, and computer program product embodiments for providing telehealth services to a group of patients while maintaining patient confidentiality. A clinician is able to interact with patients individually in a group setting, conversing with and assigning modules to each through a telehealth platform. The clinician can directly pull up a patient's electronic health record (EHR) from within the platform, while shielding it from view by patients if screen sharing is enabled. Additionally, the platform anonymizes patient names in an attendee list and chat box when screen sharing, to prevent patients from seeing other patient names and information. By the approaches disclosed herein, a clinician can interact with multiple patients at once without accidentally divulging one patient's sensitive information to another.

### CROSS-REFERENCE TO RELATED APPLICATIONS

This application claims the benefit of U.S. Provisional Application No. 63/119,373, filed Nov. 30, 2020 and entitled “PRIVACY CONTROLS FOR MANAGING GROUP TELEHEALTH SESSIONS,” which is incorporated by reference herein in its entirety.

### BACKGROUND

Clinicians and their patients are increasingly turning to telehealth solutions in order to address a variety of healthcare needs. For example, mental health counselors may use a telehealth approach in order to facilitate access to patients on a busy schedule. Primary physicians may use a telehealth approach in order to remotely diagnose patients presenting visible symptoms, and even prescribe treatments. Moreover, COVID-19 has necessitated the rapid deployment of telehealth platforms across many medical services in order to reduce the incidence of face-to-face interaction between clinicians and patients where not strictly necessary.

While these platforms can simplify access to medical services, as well as reducing certain costs of these services, the privacy and security risks associated with such online services complicate the manner in which otherwise commonplace teleconference and videoconference services are provided. Accordingly, what is needed is a telehealth platform that facilitates clinician-patient interaction while maintaining patient privacy.

### BRIEF DESCRIPTION OF THE DRAWINGS

The accompanying drawings are incorporated herein and form a part of the specification.

FIG. 1 illustrates a clinician view of a group telehealth system, in accordance with an embodiment.

FIG. 2 illustrates a clinician view of a telegroup system with access to a patient's electronic health record (EHR), in accordance with an embodiment.

FIG. 3 illustrates a clinician view of a telegroup system while sharing the clinician's screen with patients attending a session, in accordance with an embodiment.

FIG. 4 illustrates a patient view of a telegroup system, in accordance with an embodiment.

FIG. 5 illustrates a patient view of a telegroup system while a clinician is sharing their screen, in accordance with an embodiment.

FIG. 6 is a flowchart illustrating steps by which a clinician's screen can be safely shared during a telegroup session, in accordance with an embodiment.

FIG. 7 is an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for providing telehealth services to a group of patients while maintaining patient confidentiality.

Recently, telehealth platforms have appeared to provide secure portals for patients to interact with clinicians over video and chat. While robust cloud-based videoconferencing systems are in use across many different industries, the particular privacy requirements of the healthcare industry necessitate the creation of entirely new and unique videoconferencing software to eliminate the disclosure of patient information to third parties.

Telehealth platforms commonly provide rudimentary videoconferencing, screen sharing, and chat features. If a clinician wishes to access the electronic health record (EHR) for a patient, the EHR is accessed through a separate, non-integrated application. These telehealth platforms are also designed for use by a single clinician-patient pair at a given time, as attending to multiple patients concurrently presents privacy risks.

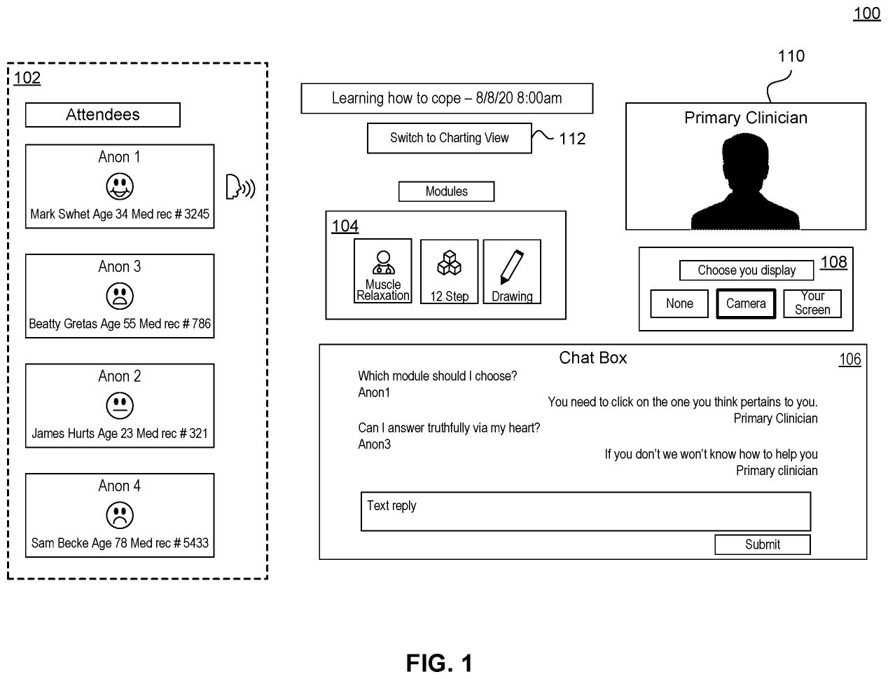

FIG. 1 illustrates a clinician view of a group telehealth system 100, in accordance with an embodiment. In this telehealth system 100, a clinician is able to interact with multiple patients concurrently. A list of these patients is shown in attendee list 102, in accordance with an embodiment. Attendee list 102 can include, by way of non-limiting example, an avatar, a live video feed, an indication of incoming audio, and personally identifying information regarding each attendee in attendee list 102.

Group telehealth system 100 also includes a module list 104, in accordance with an embodiment. Module list 104 is configured to include program modules that can be individually assigned to any of the patients in attendee list 102 by the clinician. For example, the clinician may select a ‘muscle relaxation’ module from module list 104, and assign it (e.g., through an additional prompt upon clicking a button corresponding to the module in module list 104) to a particular patient. Upon doing so, the patient will be prompted to complete the module on their device.

The clinician may repeat the process of assigning modules from module list 104 to individual patients as many times as needed, and in doing so may customize a therapy session through group telehealth system 100 to each individual patient. Because of this ability to service multiple patients simultaneously, yet individually (and while protecting individual privacy, as will be disclosed herein), group telehealth system 100 can be referred to as “telegroup” system 100 (used interchangeably hereinafter). Likewise, the process of providing and receiving customized therapy through a system like telegroup/telehealth system 100 may be termed “telegroup therapy.”

Telegroup system 100 may include a chat box 106, in accordance with an embodiment. Chat box 106 includes a text box and submission button to allow any participant, including the clinician and any patients, to interact with each other by entering text in the text box and having it appear to everyone in the chat box 106 when submitted. In an embodiment, chat messaged provided by patients are anonymized, such as with an anonymous label such as “Anon 1” or “Anon 3” (where all messages from the same patient use the same anonymous label for consistency). As shown in attendee list 102, the clinician is able to identify a specific patient by matching the anonymized label to the full personal information for the specific patient. However, other patients will not know who “Anon 1” or “Anon 3” are. This allows the clinician to conduct telegroup therapy with all of the patients at once, while allowing the patients to remain in confidence from each other while participating in the telegroup therapy session.

In additional embodiments, messages entered by patients are shared only to the clinician, and are not visible to other patients. Moreover, in another embodiment, multiple chat boxes 106 may be maintained with each individual participant.

Telegroup system 100 also includes a display selection 108, in accordance with an embodiment. Display selection 108 allows the clinician to select what should be displayed to patients in a view box corresponding to view box 110. As shown in view box 110, a view of the clinician's video camera stream is displayed as the ‘camera’ option is selected in display selection 108. This indicates that the clinician's video camera stream will be sent to the patients participating in the telegroup therapy session (i.e., the attendees in attendee list 102).

Display selection 108 may provide additional options for display to patients, including ‘none’ (turns off the video camera stream) or ‘your screen’, which shares the clinician's teleconference host screen with patients and is described in further detail below.

In accordance with an embodiment, telegroup system 100 includes an option to show private records corresponding to attendees in attendee list 102. In the exemplary embodiment of FIG. 1, charting view button 112 is configured to display the EHR for a given patient in attendee list 102 when selected.

While FIG. 1 has been described in the context of a telehealth/telegroup system 100 specifically, one skilled in the relevant arts will appreciate that similar techniques can be employed in order to facilitate the interaction by a single teleconference host (e.g., a clinician) with a plurality of teleconference attendees (e.g., patients) concurrently while protecting the confidentiality of sensitive information on the host's display as well as the privacy of individual teleconference attendees.

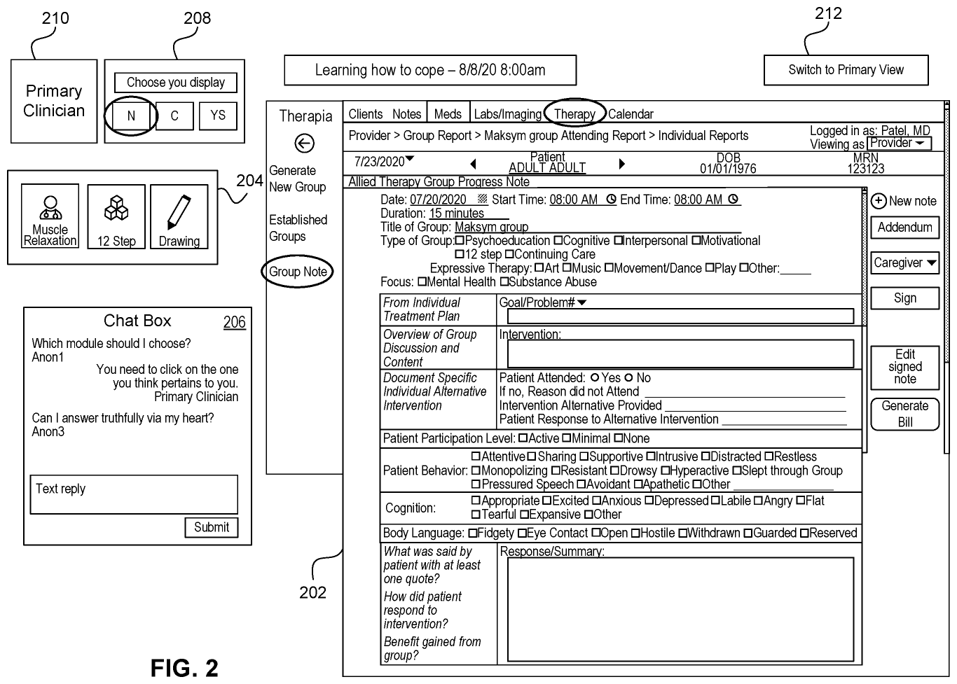

FIG. 2 illustrates a clinician view of a telegroup system 200 with access to a patient's EHR, in accordance with an embodiment. As discussed above, selection of charting view button 112 of FIG. 1 displays the EHR for a given patient in attendee list 102 of FIG. 1 when selected.

In the view of telegroup system 200, the EHR for a given patient is shown in document view 202, in accordance with an embodiment. In addition to the EHR, any sensitive document can be shown in document view 202. Document view 202 can be ‘flagged’ as sensitive information, such that if the clinician shares their screen with patients while document view 202 is visible on the screen, the contents of document view 202 are blanked (e.g., covered up or otherwise rendered unviewable) to copies of the screen shared during screen sharing.

One skilled in the relevant arts will appreciate that such a sensitive information flag, as described in the context of document view 202, can be applied to other visible elements within telegroup system 200. In an embodiment, elements within telegroup system 200 can be flagged with a sensitive information flag using, by way of non-limiting example, markup such as eXtensible Markup Language (XML).

In an embodiment, telegroup system 200 can include other elements consistent with those in telegroup system 100 of FIG. 1. For example, telegroup system 200 may optionally display module list 204 (corresponding to module list 104 of FIG. 1), chat box 206 (corresponding to chat box 106 of FIG. 1), display selection 208 (corresponding to display selection 108 of FIG. 1), and/or view box 210 (corresponding to view box 110 of FIG. 1). A primary view button 212 may also be presented in order to return the clinician back to the view of telegroup system 100 of FIG. 1.

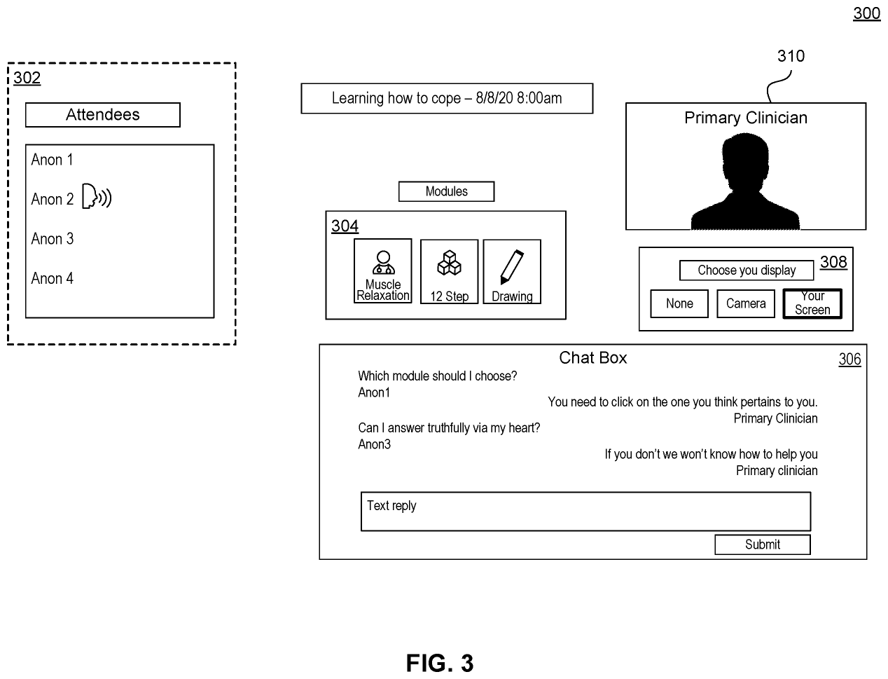

FIG. 3 illustrates a clinician view of a telegroup system 300 while sharing the clinician's screen with patients attending a session, in accordance with an embodiment. This view of telegroup system 300 tracks the view of telegroup system 100 of FIG. 1, and should largely remain the same and continue to show the same information. However, when a clinician is sharing their screen as in telegroup system 300, sensitive information must be scrubbed from view so that patients cannot inadvertently access the information of other patients.

In the exemplary embodiment shown in FIG. 3, telegroup system 300 retains several elements in common with, and unchanged from, telegroup system 100 of FIG. 1. For example, module list 304 (corresponding to module list 104 of FIG. 1), chat box 306 (corresponding to chat box 106 of FIG. 1), display selection 308 (corresponding to display selection 108 of FIG. 1), and/or view box 310 (corresponding to view box 110 of FIG. 1) remain visible on the clinician's display. Note that in the exemplary embodiment, the ‘your screen’ selection in display selection 308 is highlighted, indicating that the clinician's screen is being shared. View box 310 may be blanked out in order to avoid a hall-of-mirrors effect by showing the clinician's own screen-within-the-screen, or may be shown if the effect is preferred.

However, attendee list 302 is modified in this view to eliminate any personal information. For example, in contrast with attendee list 102 of FIG. 1, names, record numbers, ages, and video camera feeds are removed from the attendee list 302 display in screen sharing mode in order to avoid having this information presented to the other patients. In accordance with an embodiment, attendee list 302 can be flagged with the sensitive information flag applied to document view 202 of FIG. 2, whereby the element is not rendered at all while in screen sharing mode. In accordance with an embodiment, screen sharing is handled by a streaming process that renders elements as if for display on a screen, but checks whether each element has a corresponding sensitive information flag enabled before including the element in the render. One skilled in the relevant arts will appreciate that other techniques for hiding or including rendered elements within a video stream may be used, and the approaches described herein are provided by way of non-limiting example.

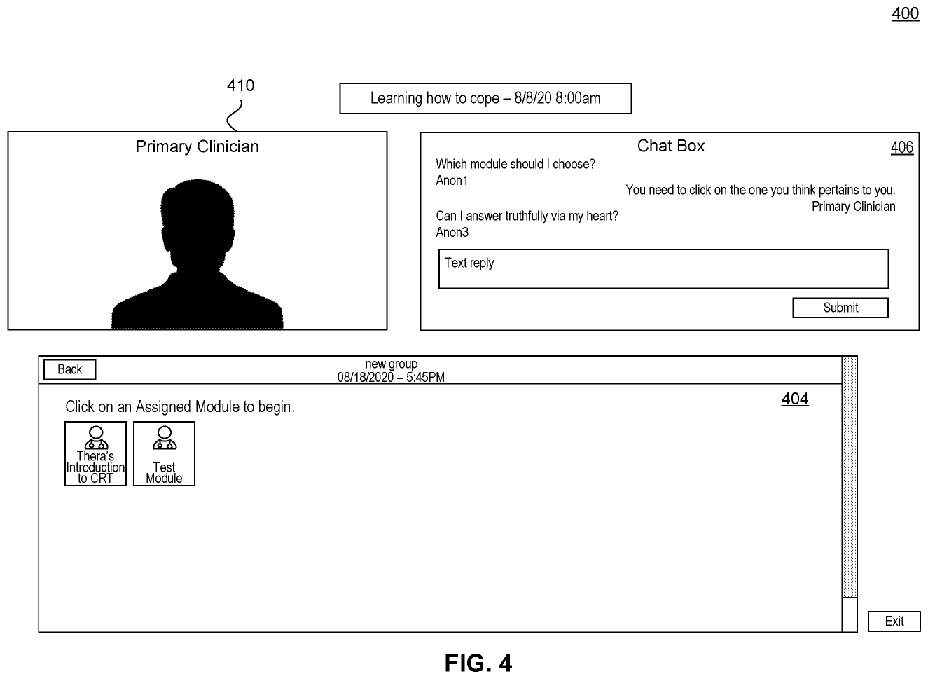

FIGS. 1-3 have shown views of the telegroup system (100, 200, and 300, respectively) from the perspective of the host of a videoconference on the telegroup system, such as a clinician. FIG. 4 illustrates a patient (client-side) view of a telegroup system 400, in accordance with an embodiment. In this view, the patient can interact with chat box 406, entering and submitting messages that will be shared anonymously with other patients, and which can be referenced with the patient information by the clinician in order to identify the patient as the source of the message (as shown in FIG. 1). Telegroup system also includes view box 410 which shows a video camera stream from the clinician.

Module box 404 is configured to display selectable modules to the patient, in accordance with an embodiment. When a clinician assigns a module to the patient (i.e., the patient whose view corresponds to telegroup system 400), the module appears as an option within module box 404. The patient may then click on a button corresponding to the module in order to open the module and complete the tasks associated with that module, in accordance with an embodiment. The module may open in a new window, full screen on the patient's display, or remain within module box 404, by way of non-limiting example.

Upon completion of the module, the completed module may be removed from the list of module box 404, and the patient may be returned to the view shown in module box 404 in order to select a new module. Additionally, when the patient completes the assigned module, the clinician may be notified (e.g., by a pop up or other message) of the patient's completion of the same.

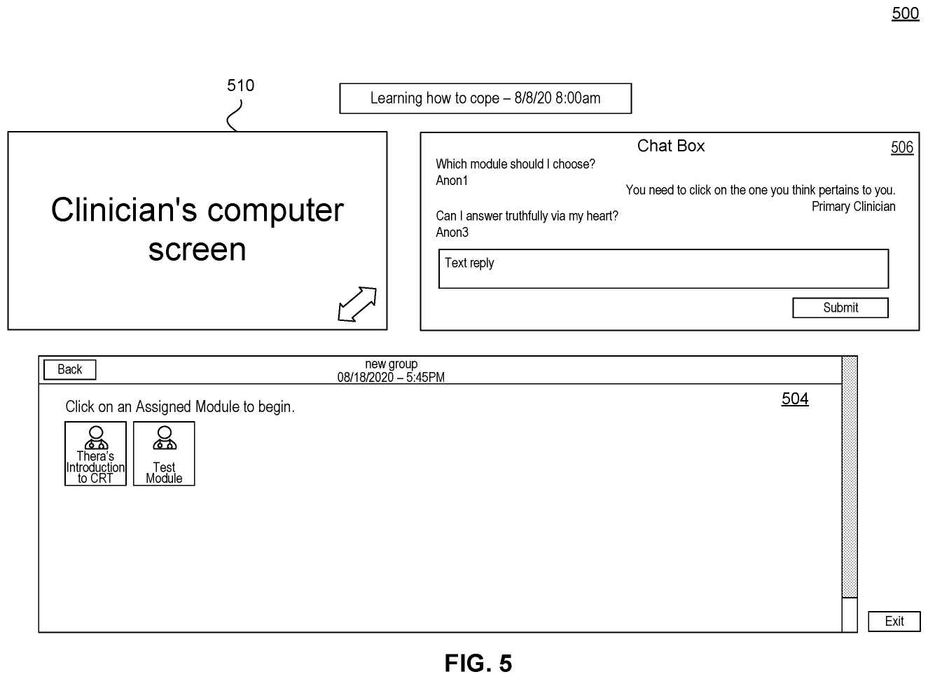

FIG. 5 illustrates a patient view of a telegroup system 500 while a clinician is sharing their screen, in accordance with an embodiment. Telegroup system 500 corresponds to the patient view of telegroup system 400 of FIG. 4, with the same module box 504 (corresponding to module box 404 of FIG. 4) and chat box 506 (corresponding to chat box 406 of FIG. 4) by way of non-limiting example. However, in telegroup system 500, view box 510 (which corresponds to view box 410 of FIG. 4) is used to display a screen share view of the clinician's computer screen in accordance with an embodiment.

The patient may optionally interact with view box 510 by selecting a ‘full screen’ (or other enlargement) option in order to more clearly see the clinician's shared screen. In accordance with an embodiment, the patient-side telegroup system 500 receives rendered screen images of the clinician's computer screen in which sensitive information has been removed and not included in the render, for display in view box 510. In accordance with an alternative embodiment, the patient-side telegroup system receives elements corresponding to elements displayed in the clinician-side telegroup system, with the sensitive information elements omitted, for rendering within view box 510. As previously noted, one skilled in the relevant arts will appreciate that other techniques for hiding or including rendered elements within a video stream may be used, and the approaches described herein are provided by way of non-limiting example.

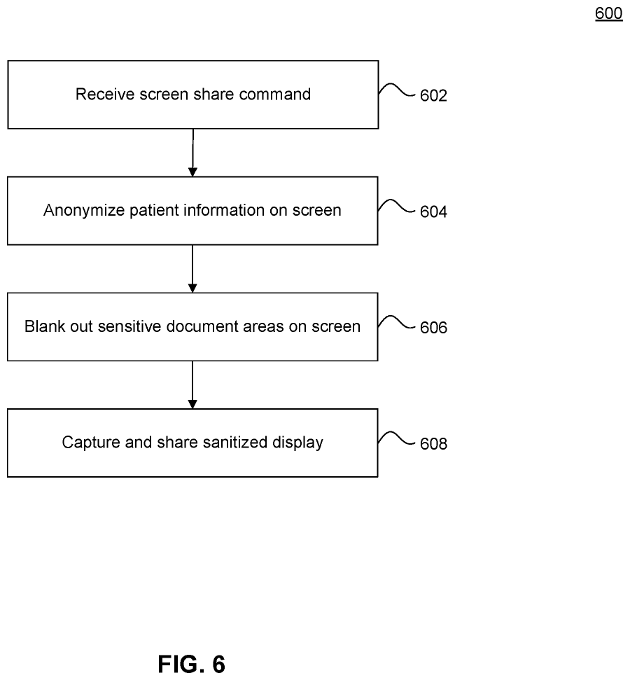

FIG. 6 is a flowchart 600 illustrating steps by which a clinician's screen can be safely shared during a telegroup session, in accordance with an embodiment. As described above with respect to the clinician-side telegroup systems 100, 200, and 300 of FIGS. 1-3, respectively, and with respect to the patient-side telegroup systems 400 and 500 of FIGS. 4 and 5, respectively, sensitive information can be ‘blanked out’ or otherwise omitted from rendering by a variety of techniques.

At step 602, a clinician-side telegroup system receives a command to share the clinician's screen with the patients attending the telegroup session (i.e., the videoconference host receives a command to share the host's screen with videoconference attendee clients). At step 604, patient information visible on screen is anonymized such as, for example, by changing the manner in which the clinician-side telegroup system is rendered to remove sensitive information from display (see, e.g., attendee list 302 of FIG. 3). At step 606, sensitive document areas are blanked out from the render (either on the clinician-side or the patient-side telegroup system, as described above by way of non-limiting example). Finally, at step 608, the sanitized display is captured and shared with each patient on the patient-side telegroup system.

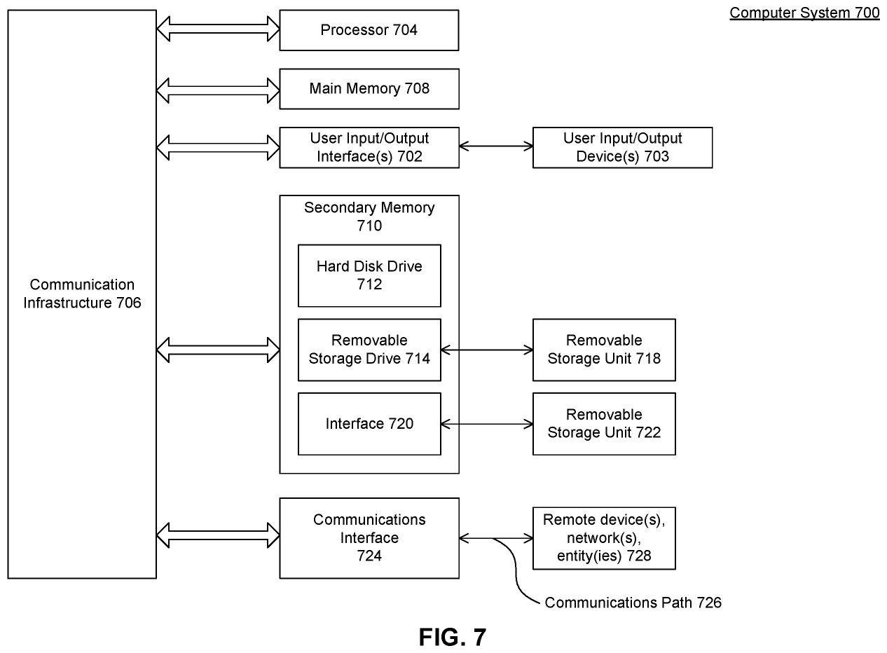

Various embodiments may be implemented, for example, using one or more well-known computer systems, such as computer system 700 shown in FIG. 7. One or more computer systems 700 may be used, for example, to implement any of the embodiments discussed herein, as well as combinations and sub-combinations thereof.

Computer system 700 may include one or more processors (also called central processing units, or CPUs), such as a processor 704. Processor 704 may be connected to a communication infrastructure or bus 706.

Computer system 700 may also include customer input/output device(s) 703, such as monitors, keyboards, pointing devices, etc., which may communicate with communication infrastructure 706 through customer input/output interface(s) 702.

One or more of processors 704 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 may also include a main or primary memory 708, such as random access memory (RAM). Main memory 708 may include one or more levels of cache. Main memory 708 may have stored therein control logic (i.e., computer software) and/or data.

Computer system 700 may also include one or more secondary storage devices or memory 710. Secondary memory 710 may include, for example, a hard disk drive 712 and/or a removable storage device or drive 714. Removable storage drive 714 may be a floppy disk drive, a magnetic tape drive, a compact disk drive, an optical storage device, tape backup device, and/or any other storage device/drive.

Removable storage drive 714 may interact with a removable storage unit 718. Removable storage unit 718 may include a computer usable or readable storage device having stored thereon computer software (control logic) and/or data. Removable storage unit 718 may be a floppy disk, magnetic tape, compact disk, DVD, optical storage disk, and/ any other computer data storage device. Removable storage drive 714 may read from and/or write to removable storage unit 718.

Secondary memory 710 may include other means, devices, components, instrumentalities or other approaches for allowing computer programs and/or other instructions and/or data to be accessed by computer system 700. Such means, devices, components, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 700 may further include a communication or network interface 724. Communication interface 724 may enable computer system 700 to communicate and interact with any combination of external devices, external networks, external entities, etc. (individually and collectively referenced by reference number 728). For example, communication interface 724 may allow computer system 700 to communicate with external or remote devices 728 over communications path 726, which may be wired and/or wireless (or a combination thereof), and which may include any combination of LANs, WANs, the Internet, etc. Control logic and/or data may be transmitted to and from computer system 700 via communication path 726.

Computer system 700 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 700 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 700 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 7. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A method, comprising:
receiving, by one or more computing devices, a screen share command from a videoconference host;
sanitizing, by the one or more computing devices, a screen of the videoconference host;
capturing, by the one or more computing devices, the sanitized screen of the videoconference host; and
sharing, by the one or more computing devices, the captured sanitized screen.

2. The method of claim 1, wherein the sanitizing the screen of the videoconference host comprises:
identifying, by the one or more computing devices, a sensitive document area on the screen of the videoconference host; and
blanking out, by the one or more computing devices, the sensitive document area on the screen of the videoconference host.

3. The method of claim 2, wherein identifying the sensitive document area comprises identifying a privacy flag associated with the sensitive document area.

4. The method of claim 1, wherein the screen of the videoconference host includes a participant list comprising a plurality of participant names, and wherein the sanitizing the screen of the videoconference host comprises:
anonymizing, by the one or more computing devices, the plurality of participant names in the participant list, wherein capturing the sanitized screen of the videoconference host captures the participant list with the anonymized plurality of participant names in the participant list.

5. The method of claim 1, further comprising:
displaying, by the one or more computing devices, a module menu configured to assign a module of the module menu by the videoconference host to a videoconference participant of a plurality of videoconference participants.

6. The method of claim 1, further comprising:
switching, by the one or more computing devices, to a view mode for a sensitive document; and
disabling, by the one or more computing devices, the sharing of the captured sanitized screen responsive to the switching to the view mode for the sensitive document.

7. The method of claim 6, wherein the sensitive document comprises an electronic health record corresponding to a videoconference participant of a plurality of videoconference participants.

8. A system, comprising:
a memory configured to store operations; and
one or more processors configured to perform the operations, the operations comprising:
receiving a screen share command from a videoconference host,
sanitizing a screen of the videoconference host,
capturing the sanitized screen of the videoconference host, and
sharing the captured sanitized screen.

9. The system of claim 8, wherein the sanitizing the screen of the videoconference host comprises:
identifying a sensitive document area on the screen of the videoconference host; and
blanking out the sensitive document area on the screen of the videoconference host.

10. The system of claim 9, wherein identifying the sensitive document area comprises identifying a privacy flag associated with the sensitive document area.

11. The system of claim 8, wherein the screen of the videoconference host includes a participant list comprising a plurality of participant names, and wherein the sanitizing the screen of the videoconference host comprises:
anonymizing the plurality of participant names in the participant list, wherein capturing the sanitized screen of the videoconference host captures the participant list with the anonymized plurality of participant names in the participant list.

12. The system of claim 8, further comprising:
displaying a module menu configured to assign a module of the module menu by the videoconference host to a videoconference participant of a plurality of videoconference participants.

13. The system of claim 8, further comprising:
switching to a view mode for a sensitive document; and
disabling the sharing of the captured sanitized screen responsive to the switching to the view mode for the sensitive document.

14. The system of claim 13, wherein the sensitive document comprises an electronic health record corresponding to a videoconference participant of a plurality of videoconference participants.

15. A computer readable storage device having instructions stored thereon, execution of which, by one or more processing devices, causes the one or more processing devices to perform operations comprising:
receiving a screen share command from a videoconference host;
sanitizing a screen of the videoconference host;
capturing the sanitized screen of the videoconference host; and
sharing the captured sanitized screen.

16. The computer readable storage device of claim 15, wherein the sanitizing the screen of the videoconference host comprises:
identifying a sensitive document area on the screen of the videoconference host; and
blanking out the sensitive document area on the screen of the videoconference host.

17. The computer readable storage device of claim 16, wherein identifying the sensitive document area comprises identifying a privacy flag associated with the sensitive document area.

18. The computer readable storage device of claim 15, wherein the screen of the videoconference host includes a participant list comprising a plurality of participant names, and wherein the sanitizing the screen of the videoconference host comprises:
anonymizing the plurality of participant names in the participant list, wherein capturing the sanitized screen of the videoconference host captures the participant list with the anonymized plurality of participant names in the participant list.

19. The computer readable storage device of claim 15, the operations further comprising:
displaying a module menu configured to assign a module of the module menu by the videoconference host to a videoconference participant of a plurality of videoconference participants.

20. The computer readable storage device of claim 15, the operations further comprising:
switching to a view mode for a sensitive document; and
disabling the sharing of the captured sanitized screen responsive to the switching to the view mode for the sensitive document.
